(19) United States
(12) Patent Application Publication
(10) Pub. No.: US 2005/0220731 A1
(43) Pub. Date: Oct. 6, 2005

(54) Nail varnish composition comprising at least one polymer and at least one plasticizer

(76) Inventors: Philippe Ilekti, Maison-Alfort (FR); Frederic Leuridan, Paris (FR)

Correspondence Address: Thomas L. Irving;FINNEGAN, HENDERSON, FARABOW,, GARRETT & DUNNER, L.L.P., 901 New York Avenue, N.W., Washington, DC, 20001-4413, US

(21) Appl. No.: 11/086,906

(22) Filed: Mar. 23, 2005

Related U.S. Application Data
(60) Provisional application No. 60/555,337, filed on Mar. 23, 2004.

(30) Foreign Application Priority Data
Mar. 23, 2004 (FR) 04 50572

(51) Int. Cl. A61K 7/04

(52) U.S. Cl. 424/61

(57) ABSTRACT

The present disclosure relates to a nail varnish composition comprising, in a cosmetically acceptable medium comprising an organic solvent medium, at least one block polymer and at least one plasticizer, such that when the composition forms a film, the film exhibits a water uptake of less than or equal to 10%. The present disclosure also relates to a process comprising applying such a composition to the nails to give a film which can be glossy, can have good holding power, and can be water-resistant on nails.

This application claims benefit of U.S. Provisional Application No. 60/555,337, filed Mar. 23, 2004, and French Application No. 04/50572, filed Mar. 23, 2004, the contents of both of which are incorporated herein by reference.

The present disclosure relates to a nail varnish composition comprising at least one block polymer and at least one plasticizer. The present disclosure also relates to a process for making up or caring for the nails.

The nail varnish composition as disclosed herein may be employed as a varnish base or base-coat, as a nail makeup product, as a finishing composition, also called topcoat, to be applied to the nail makeup product, or as a cosmetic nail care product. These compositions may be applied to the nails of human beings or else to false nails.

Compositions for the application, for example, to the nail, of nail varnish or nail care base-type in a solvent medium, are known which can comprise, conventionally, at least one film-forming polymer, optionally a plasticizer, pigments, rheological agents and solvents.

At the present time nitrocellulose still remains a principal film-former that can be widely used in solvent-based nail varnishes in formulations featuring optimized gloss and hold. Formulations comprising nitrocelluloses can allow films to be obtained with the appropriate level of hardness and gloss, but these films can lack adhesion to the nail. This drawback may be remedied by adding plasticizers; in that case, however, it can be necessary to use very large amounts of plasticizers and co-resins, of the same order as the amount of nitrocellulose.

Research has been carried out into replacing nitrocellulose by other film-forming agents, such as polyacrylics and polyurethanes, in nail varnishes, an example being the aqueous polyurethane dispersions described in the European Patent No. EP 0648485, but have not given satisfactory results, for example, in terms of hold and of resistance to external factors such as water or detergents.

For example, the sensitivity to water of the varnish film can accelerate the breakdown of the varnish film on the nails, in part because the film may dissolve in the water, because the film dulls in the presence of water, because the film is detached in the water, or because the film whitens under the action of the water.

The inventors have discovered, surprisingly, that the combination of at least one block polymer and at least one particular plasticizer can allow:
plasticization of the films without necessarily adding large amounts of internal plasticizers, while maintaining a good level of film hardness, and
effective resistance of the nail varnishes to impact and/or to flaking, and also good water resistance, and therefore an increase in the holding power of the varnishes on the nail over time. For example, the selection of plasticizers can make it possible to reduce the water uptake (water sensitivity) of varnish films and hence to increase their water resistance.

For instance, one aspect of the present disclosure is a nail varnish composition comprising, in a cosmetically acceptable medium comprising an organic solvent medium, at least one block polymer and at least one plasticizer, such that when the composition forms a film, the film exhibits a water uptake of less than or equal to 10%.

For example, when the composition forms a film, the film can exhibit a water uptake of less than or equal to 5%, for instance, less than or equal to 4%, such as less than or equal to 3%.

As used herein, the term “water uptake of the film” is understood to mean the percentage of water absorbed by the polymer film after 60 minutes of immersion in water at 25° C. (ambient temperature). The water uptake is measured for a layer of composition 300 μm thick (before drying), laid down with the aid of a 300μ applicator on a sheet of glass equipped with a layer of Teflon-coated tape and then dried at 30° C. for 24 hours on a thermostated plate. Three pieces measuring approximately 1 Cm² are cut from the dry film and then weighed (mass measurement M1), and then are immersed in water for 60 minutes; following immersion, the piece of film is wiped to removed the excess surface water and then weighed again (mass measurement M2). The difference M2−M1 corresponds to the amount of water absorbed by the film. The water uptake is equal to [(M2−M1)/M1]×100 and is expressed as a percentage by weight of water relative to the weight of the film.

The present disclosure also relates to a nail varnish composition comprising, in a cosmetically acceptable medium comprising an organic solvent medium, at least one block polymer and at least one plasticizer chosen from esters obtained from the reaction of a carboxylic acid with a diol, polyethers, dimethicone copolyols, and ethyltosylamide.

As used herein, the term “cosmetically acceptable medium” is understood to mean a non-toxic medium that can be applied to the skin, the epidermal derivatives or the lips of the face of human beings.

The present disclosure further relates to a cosmetic process for making up or non-therapeutically caring for the nails, comprising the application to the nails of at least one layer of the nail varnish composition as disclosed herein.

The present disclosure additionally relates to the use of a nail varnish composition comprising at least one block polymer and at least one plasticizer chosen from esters obtained from the reaction of a carboxylic acid with a diol, polyethers, dimethicone copolyols, ethyltosylamide and mixtures thereof to give a film applied to the nails, which can be glossy and/or have a good hold and be water resistant.

This block copolymer can be formulated as a film-forming polymer alone or complementary to a conventional film-forming polymer such as nitrocellulose, or a cellulose derivative, and may avoid, in this latter case, the need for adding large amounts of plasticizers.

1) Block Polymer

The at least one block polymer of the composition according to the present disclosure can be, for example, a film-forming linear ethylenic block polymer.

As used herein, the term “ethylenic” polymer is understood to mean a polymer obtained by polymerizing monomers comprising an ethylenic unsaturation.

As used herein, the term “block” polymer is understood to mean a polymer comprising at least 2 different blocks, such as at least 3 different blocks.

The polymer is a polymer with a linear structure, as opposed to a polymer of non-linear structure, for example, a polymer of branched, starburst or grafted structure, or the like.

As used herein, the term “film-forming” polymer is understood to mean a polymer capable of forming, by itself or in the presence of an auxiliary film-forming agent, a continuous film that adheres to a support, for instance to keratin materials.

For example, the block polymer of the composition according to the present disclosure can comprise at least one first block and at least one second block having different glass transition temperatures (Tg), wherein the at least one first and at least one second blocks are linked together via an intermediate segment comprising at least one constituent monomer of the first block and at least one constituent monomer of the second block.

For instance, the first block and at least the second block of the block polymer can be mutually incompatible.

As used herein, the term “mutually incompatible blocks” is understood to mean that the mixture formed from the polymer corresponding to the first block and from the polymer corresponding to the second block is not miscible in the organic solvent that is contained in major amount by weight in the organic solvent medium of the composition, at room temperature (25° C.) and atmospheric pressure (10⁵ Pa), when the polymer mixture is present in an amount greater than or equal to 5% by weight, relative to the total weight of the mixture (polymers and solvent), it being understood that:
i) the polymers are present in the mixture in an amount such that the respective weight ratio ranges from 10/90 to 90/10, and
ii) each of the polymers corresponding to the first and second blocks has an average (weight-average or number-average) molecular mass equal to that of the block polymer±15%.

When the organic solvent medium comprises a mixture of organic solvents, for example, if two or more solvents are present in identical mass proportions, the polymer mixture is immiscible in at least one of them. When the organic solvent medium comprises only one solvent, then it is the major solvent.

The intermediate segment, which is a block comprising at least one constituent monomer of the at least one first block and at least one constituent monomer of the at least one second block of the polymer, can make it possible to “compatibilize” these blocks.

For example, the block polymer can be made such that it does not comprise silicon atoms in its skeleton. As used herein, the term “skeleton” is understood to mean the main chain of the polymer, as opposed to the pendant side chains.

For example, the block polymer of the composition according to the present disclosure can be water-insoluble, i.e. the polymer is not soluble in water or in a mixture of water and at least one solvent chosen from linear and branched lower monoalcohols comprising from 2 to 5 carbon atoms, for instance ethanol, isopropanol or n-propanol, without pH modification, when present in an active material amount of at least 1% by weight, at room temperature (25° C.).

In one embodiment of the present disclosure, the at least one block polymer is a non-elastomeric polymer. As used herein, the term “non-elastomeric polymer” is understood to mean a polymer which, when it is subjected to a constraint intended to stretch it (for example by 30% relative to its initial length), does not return to a length substantially identical to its initial length when the constraint ceases. For example, the term “non-elastomeric polymer” can be a polymer with an instantaneous recovery Rᵢ<50% and a delayed recovery R₂ₕ<70% after having been subjected to a 30% elongation, such as, Rᵢ<30% and R₂ₕ<50%.

For instance, the non-elastomeric nature of the polymer is determined according to the following protocol: A polymer film is prepared by pouring a solution of the polymer into a Teflon-coated mold, followed by drying for 7 days in an environment conditioned at 23±5° C. and 50±10% relative humidity. A film about 100 μm thick is thus obtained, from which are cut rectangular specimens (for example using a punch) 15 mm wide and 80 mm long. This sample is subjected to a tensile stress using a machine sold under the reference Zwick, under the same temperature and humidity conditions as for the drying. The specimens are pulled at a speed of 50 mm/min and the distance between the jaws is 50 mm, which corresponds to the initial length (l₀) of the specimen.

The instantaneous recovery Rᵢ is determined in the following manner:
the specimen is pulled by 30% (εₘₐₓ), i.e. about 0.3 times its initial length (l₀)
the constraint is released by applying a return speed equal to the tensile speed, i.e. 50 mm/min, and the residual elongation of the specimen is measured as a percentage, after returning to zero filler constraint (εᵢ).

The percentage instantaneous recovery (Rᵢ) is given by the following formula:
Rᵢ=(εₘₐₓ−εᵢ)/εₘₐₓ)×100

To determine the delayed recovery, after 2 hours the rate of percentage residual elongation of the specimen (ε₂ₕ) is measured, 2 hours after returning to zero filler constraint. The percentage delayed recovery after 2 hours (R₂ₕ) is given by the following formula:
R₂ₕ=(εₘₐₓ−ε₂ₕ)/εₘₐₓ)×100

For example, in one embodiment of the present disclosure, the at least one polymer has, for instance, an instantaneous recovery Rᵢ of 10% and a delayed recovery R₂ₕ of 30%.

The at least one block polymer according to the present disclosure comprises at least one first block and at least one second block having different glass transition temperatures (Tg), wherein the at least one first and at least one second blocks are linked together via an intermediate segment comprising at least one constituent monomer of the first block and at least one constituent monomer of the second block.

It is noted that, in the text hereinabove and hereinbelow, the terms “first” and “second” blocks do not in any way condition the order of the blocks in the polymer structure.

For instance, the at least one block polymer comprised in the composition according to the present disclosure can have a polydispersity index I of greater than 2, for example ranging from 2 to 9, such as greater than or equal to 2.5, for example ranging from 2.5 to 8, suhc as greater than or equal to 2.8, and for instance ranging from 2.8 to 6.

The polydispersity index I of the polymer is equal to the ratio of the weight-average mass Mw to the number-average mass Mn.

The weight-average molar mass (Mw) and number-average molar mass (Mn) are determined by gel permeation liquid chromatography (THF solvent, calibration curve established with linear polystyrene standards, refractometric detector). The weight-average mass (Mw) of the at least one block polymer according to the present disclosure can be, for instance, less than or equal to 300,000; it can range, for example, from 35,000 to 200,000, such as from 45,000 to 150,000. The number-average mass (Mn) of the polymer according to the present disclosure can be, for instance, less than or equal to 70,000; it can range, for example, from 10,000 to 60,000, such as from 12,000 to 50,000.

Each block of the at least one block polymer of the composition according to the present disclosure is derived from one type of monomer or from several different types of monomer. This means that each block may comprise a homopolymer or a copolymer, wherein the copolymer constituting the block may in turn be random or alternating.

For example, the intermediate segment comprising at least one constituent monomer of the first block and at least one constituent monomer of the second block of the polymer can be a random polymer.

The intermediate block can be, for instance, derived essentially from constituent monomers of the at least one first block and of the at least one second block.

As used herein, the term “essentially” is understood to mean at least 85%, for instance, at least 90%, such as 95% and 100%.

The intermediate block of the at least one block polymer can have, for example, a glass transition temperature Tg that is between the glass transition temperatures of the first and second blocks.

As disclosed above, the at least one first and at least one second blocks of the at least one block polymer of the composition have different glass transition temperatures.

The glass transition temperatures indicated for the first and second blocks of the block polymer may be theoretical Tg values determined from the theoretical Tg values of the constituent monomers of each of the blocks, which may be found in a reference manual such as the Polymer Handbook, 3rd Edition, 1989, John Wiley, according to the following relationship, known as Fox's law:

$$1 / Tg = {\sum }_{i} ( {\overline{ω}}_{i} / {Tg}_{i} ) ,$$

wherein \overline{ω}ᵢ is the mass fraction of the monomer i in the block under consideration and Tgᵢ is the glass transition temperature of the homopolymer of the monomer i. Unless otherwise indicated, the Tg values indicated for the first and second blocks of the block polymer in the present patent application are theoretical Tg values.

The difference between the glass transition temperatures of the at least one first and at least one second blocks of the block polymer can be greater than 10° C., for instance, greater than 20° C., such as greater than 30° C. For example, the at least one first block of the block polymer can be chosen from:
a) a block with a Tg of greater than or equal to 40° C.,
b) a block with a Tg of less than or equal to 20° C.,
c) a block with a Tg of between 20° C. and 40° C.,
   and the at least one second block can be chosen from a category a), b) and c), with the proviso that the at least one second block is different from the at least one first block.

In the present disclosure, the expression:

“between . . . and . . . ” is intended to denote a range of values for which the limits mentioned are excluded, and the expressions “from . . . to . . . ” and “ranging from . . . to . . . ” are intended to denote a range of values for which the limits are included.

a) Block with a Tg of Greater Than or Equal to 40° C.

The block with a Tg of greater than or equal to 40° C. of the block polymer has, for example, a Tg ranging from 40° C. to 150° C., such as greater than or equal to 50° C., for example ranging from 50° C. to 120° C., such as greater than or equal to 60° C., for example ranging from 60° C. to 120° C.

The block with a Tg of greater than or equal to 40° C. may be a homopolymer or a copolymer.

In the case where this block is a homopolymer, it is derived from monomers which are such that the homopolymers prepared from these monomers have glass transition temperatures of greater than or equal to 40° C. This first block may be a homopolymer comprising only one type of monomer (for which the Tg of the corresponding homopolymer is greater than or equal to 40° C.).

In the case where the first block is a copolymer, it may be totally or partially derived from at least one monomer, the nature and amount of which are chosen such that the Tg of the resulting copolymer is greater than or equal to 40° C. The copolymer may comprise, for example:
monomers which are such that the homopolymers prepared from these monomers have Tg values of greater than or equal to 40° C., for example a Tg ranging from 40° C. to 150° C., such as greater than or equal to 50° C., for example ranging from 50° C. to 120° C., such as greater than or equal to 60° C., for example ranging from 60° C. to 120° C., and
monomers which are such that the homopolymers prepared from these monomers have Tg values of less than 40° C., chosen from monomers with a Tg of between 20 and 40° C. and/or monomers with a Tg of less than or equal to 20° C., for example a Tg ranging from −100° C. to 20° C., such as less than 15° C., for instance, ranging from −80° C. to 15° C., such as less than 10° C., for example ranging from −50° C. to 0° C., as described below.

The monomers whose homopolymers have a glass transition temperature of greater than or equal to 40° C., can be chosen, for example, from the following monomers, also known as the main monomers:
methacrylates of formula CH₂═C(CH₃)—COOR₁
   wherein R₁ is chosen from linear and branched unsubstituted alkyl groups comprising from 1 to 4 carbon atoms, such as a methyl, ethyl, propyl or isobutyl groups, and C₄ to C₁₂ cycloalkyl groups,
acrylates of formula CH₂═CH—COOR₂
   wherein R₂ is chosen from C₄ to C₁₂ cycloalkyl groups, such as isobornyl acrylate, and tert-butyl groups,
(meth)acrylamides of formula: 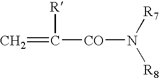
   wherein R₇ and R₈, which may be identical or different, are chosen from hydrogen atoms and linear and branched C₁ to C₁₂ alkyl groups, such as an n-butyl, t-butyl, isopropyl, isohexyl, isooctyl or isononyl group; for example, in one embodiment, R₇ is a hydrogen atom and R₈ is a 1,1-dimethyl-3-oxobutyl group; and
R′ is chosen from a hydrogen atom and methyl groups. Non-limiting examples of monomers that may be mentioned include N-butylacrylamide, N-t-butylacrylamide, N-isopropylacrylamide, N,N-dimethylacrylamide and N,N-dibutylacrylamide,
   and mixtures thereof.

Among the main monomers that may be used, further non-limiting mention may be made of methyl methacrylate, isobutyl (meth)acrylate and isobornyl (meth)acrylate, and mixtures thereof.

b) Block with a Tg of Less Than or Equal to 20° C.

The block with a Tg of less than or equal to 20° C. of the block polymer has, for example, a Tg ranging from −100° C. to 20° C., such as less than or equal to 15° C., for example ranging from −80° C. to 15° C., such as less than or equal to 10° C., for example ranging from −50 to 0° C.

The block with a Tg of less than or equal to 20° C. may be a homopolymer or a copolymer.

In the case where this block is a homopolymer, it is derived from monomers which are such that the homopolymers prepared from these monomers have glass transition temperatures of less than or equal to 20° C. This second block may be a homopolymer comprising only one type of monomer (for which the Tg of the corresponding homopolymer is less than or equal to 20° C.).

In the case where the block with a Tg of less than or equal to 20° C. is a copolymer, it may be totally or partially derived from at least one monomer, the nature and amount of which is chosen such that the Tg of the resulting copolymer is less than or equal to 20° C.

It may comprise, for example
at least one monomer whose corresponding homopolymer has a Tg of less than or equal to 20° C., for example a Tg ranging from −100° C. to 20° C., such as less than 15° C., for instance ranging from −80° C. to 15° C., such as less than 10° C., for example ranging from −50° C. to 0° C., and
at least one monomer whose corresponding homopolymer has a Tg of greater than 20° C., such as monomers with a Tg of greater than or equal to 40° C., for example a Tg ranging from 40° C. to 150° C., for instance, greater than or equal to 50° C., for example ranging from 50° C. to 120° C., such as greater than or equal to 60° C., for example ranging from 60° C. to 120° C., and/or monomers with a Tg of between 20° C. and 40° C., as described above.

For example, the block with a Tg of less than or equal to 20° C. can be a homopolymer.

The monomers whose homopolymer has a Tg of less than or equal to 20° C. can be chosen from, for example, the following monomers, or main monomers:
acrylates of formula CH₂═CHCOOR₃,
   wherein R₃ is chosen from linear and branched C₁ to C₁₂ unsubstituted alkyl groups, with the exception of the tert-butyl group, in which at least one hetero atom chosen from O, N and S atoms can be optionally intercalated,
methacrylates of formula CH₂═C(CH₃)—COOR₄,
   wherein R₄ is chosen from linear and branched C₆ to C₁₂ unsubstituted alkyl groups, in which at least one hetero atom chosen from O, N and S atoms can be optionally intercalated;
vinyl esters of formula R₅—CO—O—CH═CH₂
   wherein R₅ is chosen from linear and branched C₄ to C₁₂ alkyl groups,
vinyl alcohol and C₄ to C₁₂ alcohol ethers,
N—(C₄ to C₁₂)alkyl acrylamides, such as N-octylacrylamide,
and mixtures thereof.

Among the main monomers that can be used for the block with a Tg of less than or equal to 20° C., non-limiting mention may be made of alkyl acrylates whose alkyl chain comprises from 1 to 10 carbon atoms, with the exception of the tert-butyl group, such as methyl acrylate, isobutyl acrylate and 2-ethylhexyl acrylate, and mixtures thereof.

c) Block with a Tg of Between 20° C. and 40° C.

The block with a Tg of between 20° C. and 40° C. of the block polymer may be a homopolymer or a copolymer.

In the case where this block is a homopolymer, it is derived from monomers (or main monomers) which are such that the homopolymers prepared from these monomers have glass transition temperatures of between 20° C. and 40° C. This first block may be a homopolymer, comprising only one type of monomer (for which the Tg of the corresponding homopolymer ranges from 20° C. to 40° C.). The monomers whose homopolymer has a glass transition temperature of between 20° C. and 40° C. can be chosen from, for example, n-butyl methacrylate, cyclodecyl acrylate, neopentyl acrylate and isodecylacrylamide, and mixtures thereof.

In the case where the block with a Tg of between 20° C. and 40° C. is a copolymer, it is totally or partially derived from at least one monomer (or main monomer) whose nature and amount is chosen such that the Tg of the resulting copolymer is between 20° C. and 40° C.

For example, the block with a Tg of between 20° C. and 40° C. is a copolymer totally or partially derived from:
main monomers whose corresponding homopolymer has a Tg of greater than or equal to 40° C., for example a Tg ranging from 40° C. to 150° C., such as greater than or equal to 50° C., for example ranging from 50° C. to 120° C., such as greater than or equal to 60° C., for example ranging from 60° C. to 120° C., as described above, and/or
main monomers whose corresponding homopolymer has a Tg of less than or equal to 20° C., for example a Tg ranging from −100° C. to 20° C., such as less than or equal to 15° C., for instance ranging from −80° C. to 15° C. and such as less than or equal to 10° C., for example ranging from −50° C. to 0° C., as described above,
   wherein the monomers are chosen such that the Tg of the copolymer forming the first block is between 20° C. and 40° C.

Such main monomers can be chosen, by way of non-limiting example, from methyl methacrylate, isobornyl acrylate and methacrylate, butyl acrylate and 2-ethylhexyl acrylate, and mixtures thereof.

For instance, the amount of the block with a Tg of less than or equal to 20° C. can range from 10% to 85% by weight, such as from 20% to 70% and from 20% to 50% by weight, relative to the total weight of the polymer.

Each of the blocks of the block polymer may comprise in a small amount, at least one constituent monomer of the other block. Thus, the at least one first block of the block polymer may comprise at least one constituent monomer of the second block, and vice versa.

Each of the at least one first and/or at least one second blocks of the block polymer may optionally comprise, in addition to the monomers indicated above, at least one other monomer known as additional monomers, which are different from the main monomers mentioned above.

The nature and amount of the at least one additional monomer are chosen such that the block in which they are present has the desired glass transition temperature.

The at least one additional monomer can be chosen, for example, from:
a) hydrophilic monomers such as:
ethylenically unsaturated monomers comprising at least one carboxylic or sulphonic acid functional group, for instance acrylic acid, methacrylic acid, crotonic acid, maleic anhydride, itaconic acid, fumaric acid, maleic acid, acrylamidopropanesulphonic acid, vinylbenzoic acid, vinylphosphoric acid, and salts thereof;
ethylenically unsaturated monomers comprising at least one tertiary amine functional group, for instance 2-vinylpyridine, 4-vinylpyridine, dimethylaminoethyl methacrylate, diethylaminoethyl methacrylate and dimethylaminopropylmethacrylamide, and salts thereof;
methacrylates of formula CH₂═C(CH₃)—COOR₆
   wherein R₆ is chosen from linear and branched alkyl groups comprising from 1 to 4 carbon atoms, such as a methyl, ethyl, propyl or isobutyl groups, the alkyl group being substituted with at least one substituent chosen from hydroxyl groups (for instance 2-hydroxypropyl methacrylate and 2-hydroxyethyl methacrylate) and halogen atoms (Cl, Br, I or F), such as trifluoroethyl methacrylate;
methacrylates of formula CH₂═C(CH₃)—COOR₉,
   wherein R₉ is chosen from linear and branched C₆ to C₁₂ alkyl groups, in which at least one hetero atom chosen from O, N and S atoms can be optionally intercalated, the alkyl group being substituted with at least one substituent chosen from hydroxyl groups and halogen atoms (Cl, Br, I or F);
acrylates of formula CH₂═CHCOOR₁₀,
   wherein R₁₀ is chosen from linear and branched C₁ to C₁₂ alkyl groups substituted with at least one substituent chosen from hydroxyl groups and halogen atoms (Cl, Br, I or F), such as 2-hydroxypropyl acrylate and 2-hydroxyethyl acrylate; C₁ to C₁₂ alkyl-O—POE (polyoxyethylene) groups with repetition of the oxyethylene unit ranging from 5 to 30 times, for example methoxy-POE; and polyoxyethylene groups comprising from 5 to 30 ethylene oxide units;
b) ethylenically unsaturated monomers comprising at least one silicon atom, such as methacryloxypropyltrimethoxysilane and methacryloxypropyltris(trimethylsiloxy)silane.

Among the at least one additional monomer that may be used, non-limiting mention may be made of acrylic acid, methacrylic acid and trifluoroethyl methacrylate, and mixtures thereof.

According to one embodiment of the present disclosure, the at least one block polymer of the composition according to the present disclosure is a non-silicone polymer, i.e. a polymer free of silicon atoms.

The at least one additional monomer may be present in an amount of less than or equal to 30% by weight, for example from 1% to 30% by weight, such as from 5% to 20% by weight, for instance from 7% to 15% by weight, relative to the total weight of the at least one first and/or at least one second blocks.

For example, each of the at least one first and at least one second blocks can comprise at least one monomer chosen from (meth)acrylic acid esters, and optionally at least one monomer chosen from (meth)acrylic acid, and mixtures thereof.

Each of the at least one first and at least one second blocks can be derived, for instance, entirely from at least one monomer chosen from acrylic acid, (meth)acrylic acid esters and optionally from at least one monomer chosen from (meth)acrylic acid, and mixtures thereof.

The at least one block polymer of the composition according to the present disclosure can be, for example, a polymer free of styrene. As used herein, the term “polymer free of styrene” is understood to mean a polymer comprising less that 10% by weight, relative to the total weight of the polymer, for instance less than 5% by weight, such as less than 2% by weight, and less than 1% by weight, or not even comprising any of a styrene monomer such as styrene, styrene derivatives such as methylstyrene, chlorostyrene or chloromethylstyrene.

The at least one block polymer of the composition according to the present disclosure may be obtained by free-radical solution polymerization according to the following preparation process: A portion of the polymerization solvent is introduced into a suitable reactor and heated until the adequate temperature for the polymerization is reached (typically in a range from 60 to 120° C.). Once this temperature is reached, the constituent monomers of the at least one first block are introduced in the presence of some of the polymerization initiator. After a period of time T corresponding to a maximum degree of conversion of 90%, the constituent monomers of the second block and the rest of the initiator are introduced. The mixture is left to react for a period of time T′ (ranging from 3 to 6 hours), after which the mixture is cooled to room temperature, and the polymer dissolved in the polymerization solvent is obtained.

As used herein, the term polymerization solvent is understood to mean a solvent or a mixture of solvents. The polymerization solvent may be chosen, by way of non-limiting example, from ethyl acetate, butyl acetate, alcohols such as isopropanol, ethanol, aliphatic alkanes such as isododecane and mixtures thereof. For example, the polymerization solvent can be a mixture of butyl acetate and isopropanol or isododecane.

According to one embodiment of the present disclosure, the at least one block polymer of the composition according to the present disclosure comprises at least one first block with a Tg of greater than or equal to 40° C., as described above in section a) and at least one second block with a Tg of less than or equal to 20° C., as described above in section b).

For example, the at least one first block with a Tg of greater than or equal to 40° C. of the block polymer can be a copolymer derived from monomers which are such that the homopolymer prepared from these monomers has a glass transition temperature of greater than or equal to 40° C., such as the monomers described above.

For example, the at least one second block with a Tg of less than or equal to 20° C. is a homopolymer derived from monomers which are such that the homopolymer prepared from these monomers has a glass transition temperature of less than or equal to 20° C., such as the monomers described above.

The block with a Tg of greater than or equal to 40° C. may be present in the block polymer in an amount, for example, ranging from 20% to 90%, such as from 30% to 80% and further, for example, from 50% to 70% by weight of the polymer. The block with a Tg of less than or equal to 20° C. can be present in the block polymer in an amount, for example, ranging from 5% to 75%, such as from 15% to 50% and further, for example, from 25% to 45% by weight of the polymer.

Thus, according to one embodiment, the at least one block polymer of the composition as disclosed herein may comprise:
at least one first block with a Tg of greater than or equal to 40° C., for example, having a Tg ranging from 70° C. to 110° C., which is a methyl methacrylate/acrylic acid copolymer,
at least one second block with a Tg of less than or equal to 20° C., for example, ranging from 0° C. to 20° C., which is a methyl acrylate homopolymer, and
an intermediate block, which is a methyl methacrylate/acrylic acid/methyl acrylate copolymer.

According to another embodiment of the present disclosure, the at least one block polymer of the composition as disclosed herein may comprise:
at least one first block with a Tg of greater than or equal to 40° C., for example, ranging from 70° C. to 100° C., which is a methyl methacrylate/acrylic acid/trifluoroethyl methacrylate copolymer,
at least one second block with a Tg of less than or equal to 20° C., for example, ranging from 0° C. to 20° C., which is a methyl acrylate homopolymer, and
an intermediate block, which is a methyl methacrylate/acrylic acid/methyl acrylate/trifluoroethyl methacrylate random copolymer.

According to yet another embodiment of the present disclosure, the at least one block polymer of the composition as disclosed herein comprises at least one first block having a glass transition temperature (Tg) of between 20 and 40° C., in accordance with the blocks described in section c) above, and at least one second block having a glass transition temperature of less than or equal to 20° C., as described above in section b) above, or a glass transition temperature of greater than or equal to 40° C., as described in section a) above.

For example, the at least one first block with a Tg of between 20 and 40° C. can be present in an amount ranging from 10% to 85%, for instance, from 30% to 80%, such as from 50% to 70% by weight, relative to the weight of the polymer.

When the at least one second block is a block with a Tg of greater than or equal to 40° C., it can be present in an amount ranging from 10% to 85% by weight, such as from 20% to 70% and further, for example, from 30% to 70% by weight, relative to the weight of the polymer.

When the at least one second block is a block with a Tg of less than or equal to 20° C., it can be present in an amount ranging from 10% to 85% by weight, such as from 20% to 70% and further, for example, from 20% to 50% by weight, relative to the weight of the polymer.

According to one embodiment, the first block with a Tg of between 20° C. and 40° C. of the block polymer can be, for instance, a copolymer derived from monomers which are such that the corresponding homopolymer has a Tg of greater than or equal to 40° C., and from monomers which are such that the corresponding homopolymer has a Tg of less than or equal to 20° C.

The second block with a Tg of less than or equal to 20° C. or with a Tg of greater than or equal to 40° C. can be, for example, a homopolymer.

In another embodiment of the present disclosure, the at least one block polymer of the composition as disclosed herein comprises:
at least one first block with a Tg of between 20° C. and 40° C., for example with a Tg ranging from 25° C. to 39° C., which is a copolymer comprising at least one methyl acrylate monomer, at least one methyl methacrylate monomer and at least one acrylic acid monomer,
at least one second block with a Tg of greater than or equal to 40° C., for example ranging from 85° C. to 125° C., which is a homopolymer composed of methyl methacrylate monomers and
an intermediate block comprising at least one methyl acrylate monomer, methyl methacrylate and
an intermediate block comprising at least one methyl methacrylate, at least one acrylic acid monomer, and at least one methyl acrylate monomer.

The at least one block polymer according to the present disclosure may be present in the composition in an amount, for example, ranging from 0.1% to 60% by weight, such as from 0.5% to 50% by weight, and further, for example, from 1% to 40% by weight of dry matter of the block polymer, relative to the total weight of the composition.

Organic solvent medium

The cosmetically acceptable medium of the cosmetic composition according to the present disclosure comprises an organic solvent medium comprising at least one organic solvent.

The at least one organic solvent can be chosen from:
ketones which are liquid at ambient temperature, such as methyl ethyl ketone, methyl isobutyl ketone, diisobutyl ketone, isophorone, cyclohexanone and acetone;
alcohols which are liquid at ambient temperature, such as ethanol, isopropanol, diacetone alcohol, 2-butoxyethanol and cyclohexanol;
propylene glycol ethers which are liquid at ambient temperature, such as propylene glycol monomethyl ether, propylene glycol monomethyl ether acetate and dipropylene glycol mono-n-butyl ether;
cyclic ethers such as y-butyrolactone;
short-chain esters (comprising from 3 to 8 carbon atoms in total) such as ethyl acetate, methyl acetate, propyl acetate, isopropyl acetate, n-butyl acetate, isopentyl acetate, methoxypropyl acetate and butyl lactate;
ethers which are liquid at ambient temperature, such as diethyl ether, dimethyl ether or dichlorodiethyl ether;
alkanes which are liquid at ambient temperature, such as decane, heptane, dodecane or cyclohexane;
alkyl sulphoxides such as dimethyl sulphoxide;
aldehydes which are liquid at ambient temperature, such as benzaldehyde and acetaldehyde;
ethyl 3-ethoxypropionate;
carbonates such as propylene carbonate and dimethyl carbonate;
acetals such as methylal;
and mixtures thereof.

For example, the at least one solvent can be chosen from short-chain esters comprising from 3 to 8 carbon atoms in total, such as ethyl acetate, methyl acetate, propyl acetate, isopropyl acetate, n-butyl acetate, isopentyl acetate, methoxypropyl acetate, and butyl lactate.

The organic solvent medium can be present in an amount ranging from 10% to 95% by weight, relative to the total weight of the composition, for instance, from 15% to 80% by weight, such as from 20% to 60% by weight.

The cosmetically acceptable medium of the composition according to the present disclosure may optionally comprise an aqueous medium.

Plasticizer

The at least one plasticizer is chosen from esters obtained from the reaction of a carboxylic acid with at least one of diols, polyethers, dimethicone copolyols, and ethyltosylamides.

The esters obtained from the reaction of a carboxylic acid with a diol can be monoesters obtained from the reaction of a monocarboxylic acid of formula R₁₁COOH with a diol of formula HOR₁₂OH, wherein R₁₁ and R₁₂, which may be identical or different, are chosen from linear, branched and cyclic, saturated and unsaturated hydrocarbon chains comprising for example, from 3 to 15 carbon atoms and optionally at least one heteroatom such as N, O and S atoms.

For example, R₁₁ can be a C₃-C₅ alkyl radical such as propyl, butyl or isobutyl and R₁₂ can be a saturated linear hydrocarbon chain comprising from 5 to 10 carbon atoms.

The plasticizer can be, for instance, a monoester resulting from the reaction of isobutyric acid and octanediol, such as 2,2,4-trimethylpentane-1,3-diol, such as the TEXANOL Ester Alcohol sold by the company Eastman Chemical.

The polyethers can be chosen from, for example, those of formula (I):
H—(O—CₓH₂ₓ)ₘ—(O—C_{y}H_{2y})ₙ—OH (I)
wherein x and y, which may be identical or different, are integers ranging from 0 to 10, such as from 2 to 10, and further such as from 3 to 5, and
m and n are, independently of each other, integers ranging from 0 to 1000, such as from 0 to 100.

In one embodiment of the present disclosure, x is 3 and y is 0.

Among polyethers that conform to this formula, non-limiting mention may be made of polyethylene glycols, polypropylene glycols, polyethylene glycol-polypropylene glycol copolymers and mixtures thereof. For instance, polypropylene glycols of high molecular weight can be used, having, for example, a molecular mass ranging from 500 to 15,000, such as from 600 to 10,000, and, further, for example, polypropylene glycols having a molecular mass of 2,000 or 4,000.

The at least one plasticizer may also be chosen from dimethicone copolyols. A dimethicone copolyol is a polydimethylsiloxane polymer comprising polyether groups, such as polyoxyethylene or polyoxypropylene groups, for instance, polyoxypropylene groups, which are pendant or at the chain ends.

According to one embodiment, the dimethicone copolyols according to the present disclosure, do not include a long-chain alkyl group having more than 8 carbon atoms, such as C₈-C₂₂.

Among the dimethicone copolyols that may be used, non-limiting mention may be made of those chosen from formula (II): 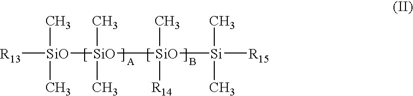
wherein:
R₁₃, R₁₄ and R₁₅, which may be identical or different, are chosen from:
   C1-C6 alkyl radicals, such as methyl, ethyl, propyl, butyl, pentyl or hexyl;
   —(CH₂)ₐ—(O—CₓH₂ₓ)ₘ—(O—C_{y}H_{2y})ₙ—O—R₁₆ groups in which
      a is an integer ranging from 0 to 8,
      R₁₆ is chosen from a hydrogen atom and C1-C6 alkyl radicals, such as methyl, ethyl, propyl, butyl, pentyl or hexyl, on condition that at least one of the radicals R₁₃, R₁₄ and R₁₅ is a group —(CH₂)ₐ—(O—CₓH₂ₓ)ₘ—(O—C_{y}H_{2y})ₙ—O—R₁₆ as defined above,
      x and y, which may be identical or different, are integers ranging from 0 to 10, for instance, from 3 to 5, such as x is 3 and y is 0,
      m and n, which may be identical or different, are integers ranging from 0 to 1000, such as from 0 to 100,
A is an integer ranging from 0 to 200;
B is an integer ranging from 0 to 100, with the proviso that A and B are not simultaneously zero.

According to one embodiment of the present disclosure, the dimethicone copolyol is chosen from the compounds of formula (II) for which
B is 0,
A is an integer ranging from 0 to 200,
R₁₃ and R₁₅ are identical and are chosen from
   —(CH₂)ₐ—(O—CₓH₂ₓ)ₘ—(O—C_{y}H_{2y})ₙ—O—R₁₆ radicals, wherein a is an integer ranging from 0 to 8, R₁₆ is chosen from a hydrogen atom and C1-C6 alkyl radicals, x is 3 and y is 0, and m and n, which may be identical or different, are integers ranging from 0 to 1000, such as from 0 to 100.

Further non-limiting mention may be made, for example, of the dimethicone copolyols comprising α,ω-propyl polyoxypropylene groups, such as the dimethicone copolyol sold under the name SILWET L-8500 by the company OSI and MAZIL.

Among the plasticizers that may be used, non-limiting mention may also be made of ethyltosylamide as sold under the name RESIMPOL 8 by the company Pan-Americana.

The at least one plasticizer may be present in an amount ranging from 1% to 15% by weight, relative to the total weight of the composition, such as from 2% to 10%, further, for example, from 3% to 8% by weight, relative to the total weight of the composition.

Optional Additional Polymers

In addition to the at least one block polymer of the composition according to the present disclosure, the composition may also comprise at least one additional polymer such as a film-forming polymer. As used herein, the term “film-forming polymer” is understood to mean a polymer capable of forming, by itself or in the presence of an auxiliary film-forming agent, a continuous film which adheres to a support, such as to keratin materials.

Among the film-forming polymers which can be used in the composition of the present disclosure, non-limiting mention may be made of synthetic polymers, of free-radical or polycondensate type, polymers of natural origin, and mixtures thereof.

The film forming polymer may be chosen from, for example cellulosic polymers such as nitrocellulose, cellulose acetate, cellulose acetobutyrate, cellulose acetopropionate, ethylcellulose, or else polyurethanes, acrylic polymers, vinyl polymers, polyvinylbutyrals, alkyd resins, resins obtained from aldehyde condensation products, such as arylsulphonamide-formaldehyde resins, such as toluenesulphonamide-formaldehyde resin, arylsulphonamide epoxy resins or else ethyltosylamide resins.

Among the film-forming polymers that may be used according to the present disclosure, non-limiting mention may be made of, for example, nitrocellulose RS ⅛ sec; RS ¼ sec; RS ½ sec.; RS 5 sec.; RS 15 sec.; RS 35 sec.; RS 75 sec.; RS 150 sec; AS ¼ sec.; AS ½ sec.; SS ¼ sec.; SS ½ sec.; SS 5 sec., sold for instance, by the company HERCULES; the toluenesulphonamide-formaldehyde resins “Ketjentflex MS80” from AKZO or “Santolite MHP”, “Santolite MS 80” from the company FACONNIER or “RESIMPOL 80” from the company PAN AMERICANA, the alkyd resin “BECKOSOL ODE 230-70-E” from the company DAINIPPON, the acrylic resin “ACRYLOID B66” from the company ROHM & HAAS, or the polyurethane resin “TRIXENE PR 4127” from the company BAXENDEN.

The at least one film-forming polymer may be present in the composition according to the present disclosure in an amount ranging from 0.1% to 60% by weight, such as from 2% to 40% by weight, and further, for example, from 5% to 25% by weight, relative to the total weight of the composition.

Colorant

The composition according to the present disclosure may also comprise at least one colorant, chosen from water-soluble dyes and pulverulent colorants, for instance pigments, nacres and flakes well known to the person skilled in the art. These colorants may be present, in the composition, in an amount ranging from 0.01% to 50% by weight, such as from 0.01% to 30% by weight, relative to the weight of the composition.

As used herein, the term “pigments” is understood to mean particles of any shape which are white or colored, mineral or organic and insoluble in the physiological medium, which are intended for coloring the composition.

As used herein, the term a “nacres” is understood to mean particles of any shape which are iridescent, such as those produced by certain molluscs in their shell, or else synthesized.

The pigments may be white or colored, mineral and/or organic. Among the mineral pigments which may be used as disclosed herein, non-limiting mention may be made of titanium dioxide, optionally surface-treated, zirconium oxide or cerium oxide, as well as zinc oxide, iron oxide (black, yellow or red), chromium oxide, manganese violet, ultramarine blue, chromium hydrate and ferric blue, metallic powders, for instance aluminium powder or copper powder. Among the organic pigments that may be used as disclosed herein, non-limiting mentioned may be made of carbon black, pigments of D & C type, and lakes based on cochineal carmine or on barium, strontium, calcium or aluminium.

The nacreous pigments may be chosen from white nacreous pigments such as mica coated with titanium or with bismuth oxychloride, colored nacreous pigments such as titanium mica coated with iron oxides, titanium mica coated with, for instance, ferric blue or chromium oxide, titanium mica coated with an organic pigment of the abovementioned type, and nacreous pigments based on bismuth oxychloride.

The water-soluble dyes include, for example, beetroot juice and methylene blue.

The composition according to the present disclosure may further comprise at least one filler, for example in an amount ranging from 0.01% to 50% by weight, relative to the total weight of the composition, such as ranging from 0.01% to 30% by weight. As used herein, the term “fillers” is understood to mean particles of any shape, colorless or white, mineral or synthetic, which are insoluble in the medium of the composition irrespective of the temperature at which the composition is manufactured. These fillers serve for instance, to modify the rheology or the texture of the composition.

The fillers may be mineral or organic of any shape, plate-like, spherical or oblong, whatever the crystallographical shape (e.g. lamellar, cubic, hexagonal, orthorhombic etc.). Among the fillers that may be used as disclosed herein, non-limiting mention may be made of talc, mica, silica, kaolin, polyamide powder (Nylon®) (Orgasol from Atochem), poly-β-alanine powder and polyethylene powder, tetrafluoroethylene polymer powders (Teflon®), lauroyllysine, starch, boron nitride, hollow polymer microspheres such as those made of polyvinylidene chloride/acrylonitrile, for instance Expancel® (Nobel Industrie), acrylic acid copolymers (Polytrap®)(Dow Corning) and silicone resin microbeads (for example Tospearls® from Toshiba), polyorgano-siloxane elastomer particles, precipitated calcium carbonate, magnesium carbonate, magnesium hydrocarbonate, hydroxyapatite, hollow silica microspheres (Silica Beads® from Maprecos), glass or ceramic microcapsules, and metal soaps derived from organic carboxylic acids comprising from 8 to 22 carbon atoms, such as from 12 to 18 carbon atoms, for example zinc, magnesium or lithium stearate, zinc laurate or magnesium myristate.

Other additives

The composition may further comprise at least one other additive commonly used in cosmetic compositions. Ingredients of this kind can be chosen from spreading agents, wetting agents, dispersants, antifoams, preservatives, UV filters, actives, surfactants, moisturizers, perfumes, neutralizing agents, stabilizers and antioxidants.

The person skilled in the art will of course take care to select the optional complementary compounds and/or the amount thereof, such that the beneficial properties of the composition according to the present disclosure are not, or are not substantially, adversely affected by the addition envisaged.

Another aspect of the present disclosure is a nail varnish product comprising: a container delimiting at least one compartment, wherein the container can be a closed container, and the compartment comprises a composition according to the present disclosure.

The container may be of any appropriate form. It may for example, be in the form of a bottle and may, at least in part, be made of a material such as glass. However, materials other than glass may be used, including, by way of example, thermoplastic materials, polypropylene, polyethylene and/or metals.

The container may be closed with a lid that can be coupled to the compartment by screwing in the closed position of the container. Alternatively the lid and the container may be coupled in a manner other than by screwing, for example, by snap fastening.

The container can be equipped with an applicator, which may be in the form of a brush composed of at least one tuft of bristles. Alternatively the applicator can be in a form other than that of a brush: for example, in the form of a spatula or a foam tip.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific example are reported as precisely as possible. Any numerical value, however, inherently contains certain errors necessarily resulting from the standard deviation found in their respective testing measurements. The following examples are intended to illustrate the invention in a non-limiting manner.

### EXAMPLES

In the examples which follow the Tgs indicated for the first and second blocks are theoretical Tgs calculated in the manner defined above.

### Example 1

### Preparation of a Polymethyl Methacrylate/Acrylic Acid/Methyl Acrylate) Polymer

100 g of butyl acetate were introduced into a 1 liter reactor, and then the temperature was increased so as to pass from room temperature (25° C.) to 90° C. in 1 hour.

100 g of methyl methacrylate, 30 g of acrylic acid, 40 g of butyl acetate, 70 g of isopropanol and 1.8 g of 2,5-bis(2-ethylhexanoylperoxy)-2,5-dimethylhexane (Trigonox® 141 from Akzo Nobel) were then added at 90° C. and over 1 hour.

The mixture was maintained for 1 hour at 90° C.

90 g of methyl acrylate, 70 g of butyl acetate, 20 g of isopropanol and 1.2 g of 2,5-bis(2-ethylhexanoylperoxy)-2,5-dimethylhexane were then introduced into the preceding mixture, still at 90° C. and over 1 hour.

The mixture was maintained for 3 hours at 90° C., then diluted with 105 g of butyl acetate and 45 g of isopropanol, and then the whole was cooled.

A solution comprising 40% polymer active material in the butyl acetate/isopropanol mixture was obtained.

A polymer comprising a poly(methyl methacrylate/acrylic acid) first block with a Tg of 100° C., a poly methyl acrylate second block with a Tg of 10° C., and an intermediate block which was a methyl methacrylate/acrylic acid/methyl polyacrylate random polymer was obtained.

This polymer had a weight-average mass of 52,000 and a number-average mass of 18,000, i.e. a polydispersity index I of 2.89.

### Example 2

### Preparation of a Poly(Methyl Methacrylate/Acrylic Acid/Methyl Acrylate) Polymer

100 g of butyl acetate were introduced into a 1 liter reactor, and then the temperature was increased so as to pass from room temperature (25° C.) to 90° C. in 1 hour.

150 g of methyl methacrylate, 30 g of acrylic acid, 30 g of methyl acrylate, 40 g of butyl acetate, 70 g of isopropanol and 1.8 g of 2,5-bis(2-ethylhexanoylperoxy)-2,5-dimethylhexane (Trigonox® 141 from Akzo Nobel) were then added at 90° C. and over 1 hour.

The mixture was maintained for 1 hour at 90° C.

90 g of methyl acrylate, 70 g of butyl acetate, 20 g of isopropanol and 1.2 g of 2,5-bis(2-ethylhexanoylperoxy)-2,5-dimethylhexane were then introduced into the preceding mixture, still at 90° C. and over 1 hour.

The mixture was maintained for 3 hours at 90° C., then diluted with 105 g of butyl acetate and 45 g of isopropanol, and then the whole is cooled.

A solution comprising 40% polymer active material in the butyl acetate/isopropanol mixture was obtained.

A polymer comprising a poly(acrylic acid/methyl acrylate) first block with a Tg of 80° C., a polymethyl acrylate second block with a Tg of 10° C., and an intermediate block which was an acrylic acid/methyl acrylate/methyl polyacrylate random polymer was obtained.

The polymer had a weight-average mass of 50,000 and a number-average mass of 17,000, i.e. a polydispersity index I of 2.95.

### Example 3

### Preparation of a Poly(Acrylic Acid/Methyl Acrylate/Trifluoroethyl Methacrylate) Polymer

100 g of butyl acetate were introduced into a 1 liter reactor, and then the temperature was increased so as to pass from room temperature (25° C.) to 90° C. in 1 hour.

120 g of methyl methacrylate, 30 g of acrylic acid, 60 g of trifluoroethyl methacrylate, 40 g of butyl acetate, 70 g of isopropanol and 1.8 g of 2,5-bis(2-ethylhexanoylperoxy)-2,5-dimethylhexane (Trigonox® 141 from Akzo Nobel) were then added at 90° C. and over 1 hour.

The mixture was maintained for 1 hour at 90° C.

90 g of methyl acrylate, 70 g of butyl acetate, 20 g of isopropanol and 1.2 g of 2,5-bis(2-ethylhexanoylperoxy)-2,5-dimethylhexane were then introduced into the preceding mixture, still at 90° C. and over 1 hour.

The mixture was maintained for 3 hours at 90° C., then diluted with 105 g of butyl acetate and 45 g of isopropanol, and then the whole was cooled.

A solution comprising 40% polymer active material in the butyl acetate/isopropanol mixture was obtained.

A polymer comprising a poly(acrylic acid/methyl methacrylate/trifluoroethyl methacrylate) first block with a Tg of 85° C., a polymethyl acrylate second block with a Tg of 10° C., and an intermediate block which was an acrylic acid/methyl acrylate/polymethyl acrylate/trifluoroethyl methacrylate random polymer was obtained.

The polymer had a weight-average mass of 53,000 and a number-average mass of 17,500, i.e. a polydispersity index I of 3.03.

### Example 4

### Preparation of a Poly(Methyl Methacrylate/Methyl Acrylate/Acrylic Acid) Polymer

210 g of ethyl acetate were introduced into a 1 liter reactor, and then the temperature was increased so as to pass from room temperature (25° C.) to 78° C. in 1 hour.

54 g of methyl methacrylate, 21 g of acrylic acid, 135 g of methyl acrylate and 1.8 g of 2,5-bis(2-ethylhexanoylperoxy)-2,5-dimethylhexane (Trigonox® 141 from Akzo Nobel) were then added at 78° C. and over 1 hour.

The mixture was maintained for 1 hour at 90° C.

90 g of methyl methacrylate, 90 g of ethyl acetate and 1.2 g of 2,5-bis(2-ethylhexanoylperoxy)-2,5-dimethylhexane were then introduced into the preceding mixture, still at 78° C. and over 1 hour.

The mixture was maintained for 3 hours at 78° C., then diluted with 150 g of ethyl acetate, and then the whole was cooled.

A solution comprising 40% polymer active material in ethyl acetate was obtained.

The polymer obtained comprised a poly(methyl acrylate/methyl methacrylate/acrylic acid) first block with a Tg of 35° C., a poly(methyl methacrylate) second block with a Tg of 100° C., and an intermediate block which was a methyl methacrylate/acrylic acid/polymethyl acrylate random polymer.

The polymer had a weight-average mass of 141,000 and a number-average mass of 50,000, i.e. a polydispersity index I of 2.82.

### Example 5

### Preparation of a Poly(Methyl Methacrylate/methyl Acrylate/Acrylic Acid) Polymer

100 g of butyl acetate were introduced into a 1 liter reactor, and then the temperature was increased so as to pass from room temperature (25° C.) to 90° C. in 1 hour.

50.4 g of methyl methacrylate, 21 g of acrylic acid, 138.6 g of methyl acrylate, 40 g of butyl acetate, 70 g of isopropanol and 1.8 g of 2,5-bis(2-ethyl-hexanoylperoxy)-2,5-dimethylhexane (Trigonox® 141 from Akzo Nobel) were then added at 90° C. and over 1 hour.

The mixture was maintained for 1 hour at 90° C.

90 g of methyl methacrylate, 70 g of butyl acetate, 20 g of isopronanol and 1.2 g of 2,5-bis(2-ethylhexanoylperoxy)-2,5-dimethylhexane were then introduced into the preceding mixture, still at 90° C. and over 1 hour.

The mixture was maintained for 3 hours at 90° C., then diluted with 105 g of butyl acetate and 45 g of isopropanol, and then the whole was cooled.

A solution comprising 40% polymer active material in the butyl acetate/isopropanol mixture was obtained.

The polymer obtained comprised a poly(methyl acrylate/methyl methacrylate/acrylic acid) first block with a Tg of 35° C., a poly(methyl methacrylate) second block with a Tg of 100° C., and an intermediate block which was a methyl methacrylate/acrylic acid/polymethyl acrylate random polymer.

### Example 6

### Nail Varnish Compositions

Nail varnishes were prepared which had the following compositions: Examples 6A to 6D include a plasticizer according to the present disclosure, Examples 6E and 6F include a comparative prior art plasticizer. All amounts are percentages.

| | Example | Example | Example | Example | Example | Example |
|---|---|---|---|---|---|---|
| Constituent | 6A | 6B | 6C | 6D | 6E | 6F |
| Polymer of Example | 25 | 25 | 25 | 25 | 25 | 25 |
| 3 (*AM) | | | | | | |
| Ester of isobutyric | 3 | | | | | |
| acid and 2,2,4- | | | | | | |
| trimethylpentane- | | | | | | |
| 1,3-diol (Texanol | | | | | | |
| from Eastman | | | | | | |
| Chemical) | | | | | | |
| Polypropylene glycol | | 3 | | | | |
| (Mw = 2000) | | | | | | |
| Dimethicone | | | 3 | | | |
| copolyol (SILWET L- | | | | | | |
| 8500 from OSI and | | | | | | |
| MAZIL) | | | | | | |
| Ethyltosylamide | | | | 3 | | |
| Hexylene glycol | | | | | 3 | |
| Polyoxyethylene/polyoxypropylene | | | | | | 3 |
| glycol | | | | | | |
| Ethyl acetate | qs 100 | qs 100 | qs 100 | qs 100 | qs 100 | qs 100 |

*AM = active material

For each of the examples the water uptake of the film of composition was measured as indicated above.

The results are presented in the following table:

| | Water uptake |
|---|---|
| Example 6A | 1.5 |
| Example 6B | 2.9 |
| Example 6C | 2 |
| Example 6D | 2.5 |
| Example 6E | 23.3 |
| Example 6F | 12 |

Compositions 6A to 6D, comprising a plasticizer according to the present disclosure, form varnish films which exhibit a lower water uptake than the comparative prior art compositions, and consequently exhibit a better water resistance.

Compositions 6A to 6D are also judged to be glossy and to have a good holding power over time.

1. A nail varnish composition comprising, in a cosmetically acceptable medium comprising an organic solvent medium, at least one block polymer and at least one plasticizer, such that when the composition forms a film, the film exhibits a water uptake of less than or equal to 10%.

2. The nail varnish composition according to claim 1, wherein the at least one plasticizer is chosen from esters obtained from the reaction of a carboxylic acid with a diol, polyethers, dimethicone copolyols, and ethyltosylamides.

3. A nail varnish composition comprising, in a cosmetically acceptable medium comprising an organic solvent medium, at least one block polymer and at least one plasticizer chosen from esters obtained from the reaction of a carboxylic acid with a diol, polyethers, dimethicone copolyols, and ethyltosylamides.

4. The nail varnish composition according to claim 3 wherein the at least one block polymer is a film-forming linear ethylenic block polymer.

5. The nail varnish composition according to claim 1, wherein the at least one block polymer comprises at least one first block and at least one second block of different glass transition temperatures (Tg), wherein the at least one first and at least one second blocks are linked together via an intermediate segment comprising at least one constituent monomer of the at least one first block and at least one constituent monomer of the at least one second block.

6. The nail varnish composition according to claim 5, wherein the at least one first and at least one second blocks are incompatible.

7. The nail varnish composition according to claim 6, wherein the at least one first block and the at least one second block of the at least one block polymer are chosen from:
a) blocks with a Tg of greater than or equal to 40° C.,
b) blocks with a Tg of less than or equal to 20° C.,
c) blocks with a Tg of between 20 and 40° C., and
with the proviso that the at least one second block is chosen from a category a), b) or c) different from the first block.

8. The nail varnish composition according to claim 7, wherein the block of the at least one block polymer with a Tg of greater than or equal to 40° C. is totally or partially derived from at least one monomer, such that the homopolymer prepared from these monomers has a glass transition temperature of greater than or equal to 40° C.

9. The nail varnish composition according to claim 8, wherein the at least one monomer whose corresponding homopolymer has a glass transition temperature of greater than or equal to 40° C. is chosen from:
methacrylates of formula CH₂═C(CH₃)—COOR₁
wherein R₁ is chosen from linear and branched unsubstituted alkyl groups comprising from 1 to 4 carbon atoms, and C₄ to C₁₂ cycloalkyl groups,
acrylates of formula CH₂═CH—COOR₂
wherein R₂ is chosen from C₄ to C₁₂ cycloalkyl groups and tert-butyl groups; and
(meth)acrylamides of formula: 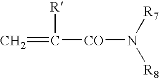
wherein R₇ and R₈, which may be identical or different, are chosen from hydrogen atoms and linear and branched alkyl groups of 1 to 12 carbon atoms; or, alternatively, R₇ is a hydrogen atom and R₈ is a 1,1-dimethyl-3-oxobutyl group, and R′ is chosen from a hydrogen atom and methyl groups.

10. The nail varnish composition according to claim 9, wherein the at least one monomer whose corresponding homopolymer has a glass transition temperature of greater than or equal to 40° C. is chosen from methyl methacrylate, isobutyl methacrylate and isobornyl (meth)acrylate.

11. The nail varnish composition according to claim 7, wherein the block of the block polymer with a Tg of less than or equal to 20° C. is totally or partially derived from at least one monomer which is such that the homopolymer prepared from the at least one monomer has a glass transition temperature of less than or equal to 20° C.

12. The nail varnish composition according to claim 11, wherein the at least one monomer whose corresponding homopolymer has a glass transition temperature of less than or equal to 20° C. is chosen from:
acrylates of formula CH₂═CHCOOR₃,
wherein R₃ is chosen from linear and branched C₁ to C₁₂ unsubstituted alkyl groups, with the exception of the tert-butyl group, in which at least one hetero atoms chosen from O, N and S atoms can be optionally intercalated,
methacrylates of formula CH₂═C(CH₃)—COOR₄,
wherein R₄ is chosen from linear and branched C₆ to C₁₂ unsubstituted alkyl groups, in which at least one hetero atom chosen from O, N and S atoms can be optionally intercalated,
vinyl esters of formula R₅—CO—O—CH═CH₂
wherein R₅ is chosen from linear and branched C₄ to C₁₂ alkyl groups,
vinyl and C₄ to C₁₂ alkyl ethers, and
N—(C₄ to C₁₂)alkyl acrylamides.

13. The nail varnish composition according to claim 12, wherein the at least one monomer whose corresponding homopolymer has a glass transition temperature of less than or equal to 20° C. is chosen from alkyl acrylates whose alkyl chain comprises from 1 to 10 carbon atoms, with the exception of the tert-butyl group.

14. The nail varnish composition according to claim 7, wherein the block with a Tg of between 20° C. and 40° C. is totally or partially derived from at least one monomer which is such that the homopolymer prepared from the at least one monomer has a glass transition temperature of between 20° C. and 40° C.

15. The nail varnish composition according to claim 7, wherein the block with a Tg of between 20° C. and 40° C. is totally or partially derived from monomers which are such that the corresponding homopolymer has a Tg of greater than or equal to 40° C., from monomers which are such that the corresponding homopolymer has a Tg of less than or equal to 20° C.

16. The nail varnish composition according to claim 7, wherein the block with a Tg of between 20 and 40° C. is totally or partially derived from at least one monomer chosen from methyl methacrylate, isobornyl acrylate, isobornyl methacrylate, trifluoroethyl methacrylate, butyl acrylate and 2-ethylhexyl acrylate.

17. The nail varnish composition according to claim 5, wherein the at least one block polymer comprises at least one first block and at least one second block, the at least one first block having a glass transition temperature (Tg) of greater than or equal to 40° C. and the at least one second block having a glass transition temperature of less than or equal to 20° C., wherein the at least one first and at least one second blocks are linked together via an intermediate segment comprising at least one constituent monomer of the first block and at least one constituent monomer of the second block.

18. The nail varnish composition according to claim 17, wherein the at least one first block of the block polymer is totally or partially derived from at least one monomer which is such that the homopolymer prepared from these monomers has a glass transition temperature of greater than or equal to 40° C.

19. The nail varnish composition according to claim 18, wherein the at least one first block is a copolymer derived from monomers which are such that the homopolymer prepared from these monomers has a glass transition temperature of greater than or equal to 40° C.

20. The nail varnish composition according to claim 18, wherein the at least one monomer whose corresponding homopolymer has a glass transition temperature of greater than or equal to 40° C. is chosen from:
methacrylates of formula CH₂═C(CH₃)—COOR₁
wherein R₁ is chosen from linear and branched unsubstituted alkyl groups comprising from 1 to 4 carbon atoms,
acrylates of formula CH₂═CH—COOR₂
wherein R₂ is chosen from C₄ to C₁₂ cycloalkyl groups and tert-butyl groups, and
(meth)acrylamides of formula: 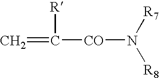
wherein R₇ and R₈, which may be identical or different, are chosen from hydrogen atoms, and linear and branched alkyl groups of 1 to 12 carbon; or, alternatively, R₇ is a hydrogen atom and R₈ is a 1,1-dimethyl-3-oxobutyl group, and R′ is chosen from a hydrogen atom and methyl groups.

21. The composition according to claim 20, wherein the at least one monomer whose corresponding homopolymer has a glass transition temperature of greater than or equal to 40° C. is chosen from methyl methacrylate, isobutyl methacrylate and isobornyl (meth)acrylate.

22. The nail varnish composition according to claim 17, wherein the at least one first block of the block polymer is present in the block polymer in an amount ranging from 20% to 90% by weight, relative to the total weight of the polymer.

23. The nail varnish composition according to claim 22, wherein the at least one first block of the block polymer is present in the block polymer in an amount ranging from 50% to 70% by weight, relative to the total weight of the polymer.

24. The nail varnish composition according to claim 17, wherein the at least one second block of the block polymer is totally or partially derived from at least one monomer which is such that the homopolymer prepared from these monomers has a glass transition temperature of less than or equal to 20° C.

25. The nail varnish composition according to claim 24, wherein the at least one second block is a homopolymer derived from monomers which are such that the homopolymer prepared from these monomers has a glass transition temperature of less than or equal to 20° C.

26. The nail varnish composition according to claim 24, wherein the at least one monomer whose corresponding homopolymer has a glass transition temperature of less than or equal to 20° C. is chosen from:
acrylates of formula CH₂═CHCOOR₃,
wherein R₃ is chosen from linear and branched C₁ to C₁₂ unsubstituted alkyl groups, with the exception of the tert-butyl group, in which at least one hetero atom chosen from O, N and S atoms can be optionally intercalated,
methacrylates of formula CH₂═C(CH₃)—COOR₄,
wherein R₄ is chosen from linear and branched C₆ to C₁₂ unsubstituted alkyl groups, in which at least one hetero atom chosen from O, N and S atoms can be optionally intercalated,
vinyl esters of formula R₅—CO—O—CH═CH₂
wherein R₅ is chosen from linear and branched C₄ to C₁₂ alkyl groups,
vinyl alcohol and C₄ to C₁₂ alcohol ethers, and
N—(C₄ to C₁₂)alkyl acrylamides.

27. The nail varnish composition according to claim 26, wherein the at least one monomer whose corresponding homopolymer has a glass transition temperature of less than or equal to 20° C. is chosen from alkyl acrylates whose alkyl chain comprises from 1 to 10 carbon atoms, with the exception of the tert-butyl group.

28. The nail varnish composition according to claim 17, wherein the at least one second block with a Tg of less than or equal to 20° C. is present in the block polymer in an amount ranging from 5% to 75% by weight, relative to the weight of the block polymer.

29. The nail varnish composition according to claim 28, wherein the at least one second block with a Tg of less than or equal to 20° C. is present in the block polymer in an amount ranging from 25% to 45% by weight, relative to the weight of the block polymer.

30. The nail varnish composition according to claim 1, wherein the at least one block polymer comprises at least one first block and at least one second block, the at least one first block having a glass transition temperature (Tg) of between 20° C. and 40° C. and the at least one second block having a glass transition temperature of less than or equal to 20° C., or a glass transition temperature of greater than or equal to 40° C., wherein the at least one first and at least one second blocks are linked together via an intermediate segment comprising at least one constituent monomer of the at least one first block and at least one constituent monomer of the at least one second block.

31. The nail varnish composition according to claim 30, wherein the at least one first block with a Tg of between 20° C. and 40° C. of the block polymer is totally or partially derived from at least one monomer which is such that the homopolymer prepared from these monomers has a glass transition temperature of between 20° C. and 40° C.

32. The nail varnish composition according to claim 30, wherein the at least one first block with a Tg of between 20° C. and 40° C. of the block polymer is a copolymer derived from monomers which are such that the corresponding homopolymer has a Tg of greater than or equal to 40° C. and from monomers which are such that the corresponding homopolymer has a Tg of less than or equal to 20° C.

33. The nail varnish composition according to claim 30, wherein the at least one first block with a Tg of between 20° C. and 40° C. of the block polymer is derived from at least one monomer chosen from methyl methacrylate, isobornyl acrylate, isobornyl methacrylate, butyl acrylate and 2-ethylhexyl acrylate.

34. The nail varnish composition according to claim 30, wherein the at least one first block with a Tg of between 20° C. and 40° C. is present in the block polymer in an amount ranging from 10% to 85% by weight, relative to the weight of the block polymer.

35. The nail varnish composition according to claim 34, wherein the at least one first block with a Tg of between 20° C. and 40° C. is present in the block polymer in an amount ranging from 50% to 70% by weight, relative to the weight of the block polymer.

36. The nail varnish composition according to claim 30, wherein the at least one second block of the block polymer has a Tg of greater than or equal to 40° C. and is totally or partially derived from at least one monomer which is such that the homopolymer prepared from these monomers has a glass transition temperature of greater than or equal to 40° C.

37. The nail varnish composition according to claim 36, wherein the at least one second block of the block polymer has a Tg of greater than or equal to 40° C. and is a homopolymer derived from monomers which are such that the homopolymer prepared from these monomers has a glass transition temperature of greater than or equal to 40° C.

38. The nail varnish composition according to claim 36, wherein the at least one monomer whose corresponding homopolymer has a glass transition temperature of greater than or equal to 40° C. is chosen from:
methacrylates of formula CH₂═C(CH₃)—COOR₁
wherein R₁ is chosen from linear and branched unsubstituted alkyl groups comprising from 1 to 4 carbon atoms, and C₄ to C₁₂ cycloalkyl groups,
acrylates of formula CH₂═CH—COOR₂
wherein R₂ is chosen from C₄ to C₁₂ cycloalkyl groups and tert-butyl groups, and
(meth)acrylamides of formula: 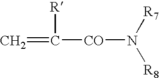
wherein R₇ and R₈, which may be identical or different, are chosen from hydrogen atoms and linear and branched alkyl groups of 1 to 12 carbon atoms; or, alternatively R₇ is a hydrogen atom, and R₈ is a 1,1-dimethyl-3-oxobutyl group, and R′ is chosen from a hydrogen atom and methyl groups.

39. The nail varnish composition according to claim 38, wherein the at least one monomer whose corresponding homopolymer has a glass transition temperature of greater than or equal to 40° C. is chosen from methyl methacrylate, isobutyl methacrylate and isobornyl (meth)acrylate.

40. The nail varnish composition according to claim 30, wherein the at least one second block with a Tg of greater than or equal to 40° C. of the block polymer is present in an amount ranging from 10% to 85% by weight, relative to the weight of the block polymer.

41. The nail varnish composition according to claim 40, wherein the at least one second block with a Tg of greater than or equal to 40° C. of the block polymer is present in an amount ranging from 30% to 70% by weight, relative to the weight of the block polymer.

42. The composition according to claim 30, wherein the at least one second block has a Tg of less than or equal to 20° C. and is totally or partially derived from at least one monomers which is such that the homopolymer prepared from the at least one monomers has a glass transition temperature of less than or equal to 20° C.

43. The nail varnish composition according to claim 42, wherein the at least one second block of the block polymer has a Tg of less than or equal to 20° C. and is a homopolymer derived from monomers which are such that the homopolymer prepared from these monomers has a glass transition temperature of less than or equal to 20° C.

44. The nail varnish composition according to claim 42, wherein the at least one monomer whose corresponding homopolymer has a glass transition temperature of less than or equal to 20° C. is chosen from:
acrylates of formula CH₂═CHCOOR₃,
wherein R₃ is chosen from linear and branched C₁ to C₁₂ unsubstituted alkyl groups, with the exception of the tert-butyl group, in which at least one hetero atoms chosen from O, N and S atoms can be optionally intercalated,
methacrylates of formula CH₂═C(CH₃)—COOR₄,
wherein R₄ is chosen from linear and branched C₆ to C₁₂ unsubstituted alkyl groups, in which at least one hetero atom chosen from O, N and S atoms can be optionally intercalated,
vinyl esters of formula R₅—CO—O—CH═CH₂
wherein R₅ is chosen from linear and branched C₄ to C₁₂ alkyl groups,
vinyl and C₄ to C₁₂ alkyl ethers; and
N—(C₄ to C₁₂)alkyl acrylamides.

45. The nail varnish composition according to claim 44, wherein the at least one monomer whose homopolymers have glass transition temperatures of less than or equal to 20° C. is chosen from alkyl acrylates whose alkyl chain comprises from 1 to 10 carbon atoms, with the exception of the tert-butyl group.

46. The nail varnish composition according to claim 30, wherein the block with a glass transition temperature of less than or equal to 20° C. is present in the block polymer in an amount ranging from 20% to 90% by weight, relative to the weight of the block polymer.

47. The nail varnish composition according to claim 46, wherein the block with a glass transition temperature of less than or equal to 20° C. is present in the block polymer in an amount ranging from 50% to 70% by weight, relative to the weight of the block polymer.

48. The nail varnish composition according to claim 5, wherein the at least one first block and/or the at least one second block of the block polymer comprises at least one additional monomer.

49. The nail varnish composition according to claim 48, wherein the at least one additional monomer is chosen from hydrophilic monomers and ethylenically unsaturated monomers that comprise at least one silicon atom.

50. The nail varnish composition according to claim 49, wherein the at least one additional monomer is chosen from:
ethylenically unsaturated monomers comprising at least one functional group chosen from carboxylic and sulphonic acid functional groups,
methacrylates of formula CH₂═C(CH₃)—COOR₆
wherein R₆ is chosen from linear and branched alkyl groups comprising from 1 to 4 carbon atoms, the alkyl group being substituted with at least one substituent chosen from hydroxyl groups and halogen atoms,
methacrylates of formula CH₂═C(CH₃)—COOR₉,
wherein R₉ is chosen from linear and branched C₆ to C₁₂ alkyl groups in which at least one hetero atom chosen from O, N and S atoms can be optionally intercalated, the alkyl group being substituted with at least one substituent chosen from hydroxyl groups and halogen atoms;
acrylates of formula CH₂═CHCOOR₁₀,
wherein R₁₀ is chosen from linear and branched C₁ to C₁₂ alkyl groups substituted with at least one substituent chosen from hydroxyl groups and halogen atoms; C₁ to C₁₂ alkyl-O—POE (polyoxyethylene) groups with repetition of the oxyethylene unit from 5 to 30 times, and polyoxyethylenated groups comprising from 5 to 30 ethylene oxide units, and
ethylenically unsaturated monomers comprising at least one tertiary amine functional group.

51. The nail varnish composition according to claim 50, wherein the at least one additional monomer is chosen from acrylic acid, methacrylic acid, and trifluoroethyl methacrylate.

52. The nail varnish composition according to claim 48, wherein the at least one additional monomer is present in an amount ranging from 1% to 30% by weight, relative to the total weight of the at least one first and/or at least one second blocks of the block polymer.

53. The nail varnish composition according to claim 48, wherein each of the at least one first and at least one second blocks of the block polymer comprise at least one monomer chosen from (meth)acrylic acid esters and optionally at least one monomer chosen from (meth)acrylic acid, and mixtures thereof.

54. The nail varnish composition according to claim 53, wherein each of the at least one first and at least one second block of the block polymer is totally derived from at least one monomer chosen from acrylic acid, (meth)acrylic acid esters and (meth)acrylic acid.

55. The nail varnish composition according to claim 5, wherein the difference between the glass transition temperatures (Tg) of the at least one first and at least one second blocks of the block polymer is greater than 10° C.

56. The nail varnish composition according to claim 55, wherein the difference between the glass transition temperatures (Tg) of the at least one first and at least one second blocks of the block polymer is greater than 40° C.

57. The nail varnish composition according to claim 5, wherein the intermediate block of the at least one block polymer has a glass transition temperature between the glass transition temperatures of the at least one first and at least one second blocks.

58. The nail varnish composition according to claim 1, wherein the at least one block polymer has a polydispersity index I of greater than 2.

59. The nail varnish composition according to claim 58, wherein the at least one block polymer has a polydispersity index I of greater than or equal to 2.8.

60. The nail varnish composition according to claim 59, wherein the block polymer has a polydispersity index ranging from 2.8 to 6.

61. The nail varnish composition according to claim 1, wherein the at least one block polymer is a film-forming linear ethylenic block polymer.

62. The nail varnish composition according to claim 1, wherein the at least one block polymer has a weight-average mass (Mw) of less than or equal to 300,000.

63. The nail varnish composition according to claim 62, wherein the at least one block polymer has a weight-average mass (Mw) ranging from 35,000 to 200,000.

64. The nail varnish composition according to claim 63, wherein the at least one block polymer has a weight-average mass (Mw) ranging from 45,000 to 150,000.

65. The nail varnish composition according to claim 1, wherein the at least one block polymer has a number-average mass (Mn) of less than or equal to 70,000.

66. The nail varnish composition according to claim 65, wherein the at least one block polymer has a number-average mass (Mn) ranging from 10,000 to 60,000.

67. The nail varnish composition according to claim 66, wherein the at least one block polymer has a number-average mass (Mn) ranging from 12,000 to 50,000.

68. The nail varnish composition according to claim 1, wherein the at least one block polymer is not soluble in an active material amount of at least 1% by weight in water or in a mixture of water and at least one linear and branched lower monoalcohol having from 2 to 5 carbon atoms, without modification of pH, at room temperature (25° C.).

69. The nail varnish composition according to claim 1, wherein the block polymer is not an elastomeric polymer.

70. The nail varnish composition according to claim 1, wherein the at least one block polymer is present in a dry matter amount ranging from 0.1% to 60% by weight, relative to the total weight of the composition.

71. The nail varnish composition according to claim 70, wherein the at least one block polymer is present in a dry matter amount ranging from 1 to 40% by weight, relative to the total weight of the composition.

72. The nail varnish composition according to claim 1, wherein the organic solvent medium comprises at least one organic solvent chosen from:
ketones which are liquid at ambient temperature;
alcohols which are liquid at ambient temperature;
propylene glycol ethers which are liquid at ambient temperature;
cyclic ethers;
short-chain esters (having 3 to 8 carbon atoms in total);
ethers which are liquid at ambient temperature;
alkanes which are liquid at ambient temperature;
alkyl sulphoxides;
aldehydes which are liquid at ambient temperature;
ethyl 3-ethoxypropionate;
carbonates; and
acetals.

73. The nail varnish composition according to claim 1, wherein the organic solvent medium is present in an amount ranging from 10% to 95% by weight, relative to the total weight of the composition.

74. The nail varnish composition according to claim 73, wherein the organic solvent medium is present in an amount ranging from 20% to 60% by weight, relative to the total weight of the composition.

75. The nail varnish composition according to claim 2, wherein the at least one plasticizer is a monoester.

76. The nail varnish composition according to claim 2, wherein the at least one plasticizer is chosen from the esters of carboxylic acid and diol obtained from reaction of a monocarboxylic acid of formula R₁₁COOH with a diol of formula HOR₁₂OH
wherein R₁₁ and R₁₂, which may be identical or different, are chosen from linear, branched and cyclic, saturated and unsaturated hydrocarbon chains comprising from 3 to 15 carbon atoms and optionally at least one hetero atom.

77. The nail varnish composition according to claim 76, wherein R₁₁ is chosen from C₃-C₅ alkyl radicals and R₁₂ is chosen from saturated linear hydrocarbon chains comprising from 5 to 10 carbon atoms.

78. The nail varnish composition according to claim 75, wherein the at least one plasticizer is a monoester resulting from the reaction of isobutyric acid and 2,2,4-trimethylpentane-1,3-diol.

79. The nail varnish composition according to claim 2, wherein the polyethers are chosen from those of formula (I):
H—(O—CₓH₂ₓ)ₘ—(O—C_{y}H_{2y})ₙ—OH (I)
wherein x and y, which may be identical or different, are integers ranging from 0 to 10, and m and n, which may be identical or different, are integers ranging from 0 to 1000.

80. The nail varnish composition according to claim 79, wherein the polyethers are chosen from those of formula (I):
H—(O—CₓH₂ₓ)ₘ—(O—C_{y}H_{2y})ₙ—OH (I)
wherein x and y, which may be identical or different, are integers ranging from 3 to 5, and m and n, which may be identical or different, are integers ranging from 0 to 100.

81. The nail varnish composition according to claim 79, wherein the polyethers are chosen from polyethylene glycols, polypropylene glycols, polyethylene glycol-polypropylene glycol copolymers and mixtures thereof.

82. The nail varnish composition according to claim 2, wherein the polyethers are chosen from polyethylene glycols and polypropylene glycols having a molecular mass ranging from 500 to 15,000, and mixtures thereof.

83. The nail varnish composition according to claim 82, wherein the polyethers are chosen from polyethylene glycols and polypropylene glycols having a molecular mass ranging from 600 to 10,000, and mixtures thereof.

84. The nail varnish composition according to claim 2, wherein the dimethicone copolyols are chosen from those of formula (II): 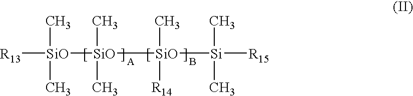
wherein:
R₁₃, R₁₄ and R₁₅, which may be identical or different, are chosen from:
C1-C6 alkyl radicals, and
—(CH₂)ₐ—(O—CₓH₂ₓ)ₘ—(O—C_{y}H_{2y})ₙ—O—R₁₆ groups in which
a is an integer ranging from 0 to 8,
R₁₆ is chosen from a hydrogen atom and C1-C6 alkyl radicals,
x and y, which may be identical or different, are integers ranging from 0 to 10,
m and n, which may be identical or different, are integers ranging from 0 to 1,000,
A is an integer ranging from 0 to 200;
B is an integer ranging from 0 to 100, with the proviso that A and B are not simultaneously zero, and
on condition that at least one of the radicals R₁₃, R₁₄ and R₁₅ is a group —(CH₂)ₐ—(O—CₓH₂ₓ)ₘ—(O—C_{y}H_{2y})ₙ—O—R₁₆ as defined above.

85. The nail varnish composition according to claim 84, wherein x is equal to 3, y is equal to zero, and m and n range from 0 to 100.

86. The nail varnish composition according to claim 84, wherein:
B is equal to zero,
A is an integer ranging from 0 to 200,
R₁₃ and R₁₅ are identical and are
—(CH₂)ₐ—(O—CₓH₂ₓ)ₘ—(O—C_{y}H_{2y})ₙ—O—R₁₆ groups in which a is an integer ranging from 0 to 8, R₁₆ is chosen from a hydrogen atom and a C1-C6 alkyl radicals, x is equal to 3 and y is equal to zero, and m and n, which may be identical or different, are integers ranging from 0 to 1000.

87. The nail varnish composition according to claim 86, wherein m and n, which may be identical or different, are integers ranging from 0 to 100.

88. The nail varnish composition according claim 84, wherein the at least one plasticizer is a dimethicone copolyol containing α,ω-propyl polyoxypropylene groups.

89. The nail varnish composition according to claim 1, wherein the at least one plasticizer is present in an amount ranging from 1% to 15% by weight, relative to the total weight of the composition.

90. The nail varnish composition according to claim 89, wherein the at least one plasticizer is present in an amount ranging from 3% to 8% by weight, relative to the total weight of the composition.

91. The nail varnish composition according to claim 1, further comprising at least one additional polymer, wherein the at least one additional polymer is film-forming.

92. The nail varnish composition according to claim 91, wherein the at least one additional polymer, which is a film-forming polymer, is present in an amount ranging from 0.1% to 60% by weight, relative to the total weight of the composition.

93. The nail varnish composition according to claim 92, wherein the at least one additional polymer, which is a film-forming polymer, is present in an amount ranging from 5% to 25% by weight, relative to the total weight of the composition.

94. The nail varnish composition according to claim 1, wherein it further comprises at least one colorant.

95. The nail varnish composition according to claim 94, wherein the at least one colorant is present in an amount ranging from 0.01% to 50% by weight, relative to the weight of the composition.

96. The nail varnish composition according to claim 95, wherein the at least one colorant is present in an amount ranging from 0.01% to 30% by weight, relative to the weight of the composition.

97. A cosmetic process for making up and/or non-therapeutically caring for the nails, comprising the application to the nails of at least one layer of a nail varnish composition comprising, in a cosmetically acceptable medium comprising an organic solvent medium, at least one block polymer and at least one plasticizer, such that when the composition forms a film, the film exhibits a water uptake of less than or equal to 10%,
wherein the at least one block polymer and at least one plasticizer are present in an effective amount to make-up and/or non-therapeutically care for the nails.

98. A cosmetic kit comprising:
a container delimiting at least one compartment, the container being closed by a lid, and wherein the compartment comprises a composition comprising, in a cosmetically acceptable medium comprising an organic solvent medium, at least one block polymer and at least one plasticizer, such that when the composition forms a film, the film exhibits a water uptake of less than or equal to 10%.

99. The cosmetic kit according to claim 98, wherein the container is comprised, at least in part, of glass.

100. The cosmetic kit according to claim 98, wherein the container is comprised, at least in part, of at least one material other than glass.

101. The cosmetic kit according to claim 98, wherein, when the container is closed, the lid is screwed onto the container.

102. The cosmetic kit according to claim 98, wherein, when the container is in the closed position, the closing lid is coupled to the container other than by screwing.

103. The cosmetic kit according to claim 98, wherein it comprises an applicator in the form of a brush comprising at least one tuft of bristles.

104. The cosmetic kit according to claim 98, wherein it comprises an applicator other than a brush.
